(19) United States
(12) Patent Application Publication
(10) Pub. No.: US 2011/0229957 A1
(43) Pub. Date: Sep. 22, 2011

(54) POLYMER-MEDIATED ELECTROMAGNETIC FIELD-BASED PARTICLE CONCENTRATOR

(75) Inventors: H. Daniel Ou-Yang, Bethlehem, PA (US); Joseph Robert Junio, Freemansburg, PA (US)

(73) Assignee: LEHIGH UNIVERSITY, Bethlehem, PA (US)

(21) Appl. No.: 13/050,516

(22) Filed: Mar. 17, 2011

Related U.S. Application Data
(60) Provisional application No. 61/314,803, filed on Mar. 17, 2010.

(51) Int. Cl. C12N 7/02; C12N 1/20; C07H 21/04; C07H 21/02; C07K 1/24

(52) U.S. Cl. 435/239; 435/252.1; 536/23.1; 530/412

(57) ABSTRACT

There remains an unmet need for methods to efficiently concentrate and isolate small particles, such as nano-sized and micron-sized particles. The methods herein provide means to fulfill this need through the combination of two independent particle-concentrating mechanisms, yielding unprecedented control, confinement, and concentration enhancement ability. The method is carried out in a reversible and spatially controllable manner. Useful applications include diagnostics and bioparticle separation, material science and research uses, among other uses.

### CROSS-REFERENCE TO RELATED APPLICATIONS

This application claims the priority of U.S. Provisional Patent Application No. 61/314,803, filed on Mar. 17, 2010, which is incorporated herein by reference in its entirety.

Not Applicable

### BACKGROUND

There is a continuing and unmet demand for methods for concentrating nano and micron particles, such as for biomedical, diagnostic, bioparticle separation, and other applications involving control and isolation of such small particles.

### SUMMARY

Provided herein are methods for concentrating nano and micron particles in a reversible and spatially controllable manner, such as for biomedical diagnostic, bioparticle separation, and other applications.

In one embodiment, methods are provided for concentrating a sample, the method comprising the steps of: a) providing a sample comprising particles, the particles being provided in at least two differing sizes, the sample further comprising a polymer suitable for suspension of the particles; b) applying a first particle concentrating mechanism; and c) applying a second particle concentrating mechanism. In this embodiment, at least one of the particle concentrating mechanisms involves electromagnetic force, and at least one of the particle concentrating mechanisms involves polymer-induced depletion force. Other embodiments provide, for example, that the particles include bioparticles, that the polymer be biocompatible.

### BRIEF DESCRIPTION OF THE DRAWINGS

FIG. 1 is a graph representing data showing the relative concentration enhancement as a function of added polymer, at a fixed electromagnetic field strength in accordance herewith.

FIG. 2 illustrates 190 nm polystyrene particles mixed with 23 nm polystyrene particles before an optical confinement field is administered in accordance herewith.

FIG. 3 is a graph depicting the concentration enhancement of 210 nm polystyrene particles mixed with different PEO (polyethylene-oxide) polymer concentrations, as a function of optical field strength in accordance herewith

FIG. 4 illustrates an enhanced concentration of 190 nm polystyrene latex particles mixed with PEO polymers in a localized electric field in accordance herewith.

FIG. 5 is an illustration of depletion attraction of a hard sphere model.

FIG. 6 is a graph illustrating the mean 190 nm particle number density inside the optical bottle as a function of optical gradient-generated pressure in accordance herewith.

FIG. 7 is a graph illustrating the osmotic compressibility for 190 nm colloids at 3.75 mm, shown for 0% and 8.44% added polymer concentrations in accordance herewith.

FIG. 8 is a graph illustrating the second virial coefficients shown in Table 1 in accordance herewith.

FIG. 9 is a graph illustrating the mean 190 nm particle number density inside the optical bottle as a function of optical gradient-generated pressure for samples containing different concentrations of polymer using identical laser power in accordance herewith.

FIG. 10 is a graph illustrating the osmotic compressibility for deionized 190 nm colloids as a function of volume fraction shown at different added polymer concentrations in accordance herewith.

FIG. 11 is a graph illustrating the second virial coefficient determined from the compressibility plots in FIG. 10 in accordance herewith.

### DETAILED DESCRIPTION

Using the methods provided herein, we have demonstrated that particles of nano size (“nanoparticles” as used herein means particles between about 1 to about 900 nm in size) and micron size (e.g. between about 1 and about 1.5 microns in size) in a polymer suspension can be confined and concentrated in a highly localized region. In preferred embodiments, the nanoparticles are less than about 500 nm in size, and the microparticles are less than about 10 microns in size. More preferably, the nanoparticles are less than about 300 nm in size, and the microparticles are between about 1 to about 2 microns in size.

Inter-particle repulsion limits the degree to which particles can be concentrated with electromagnetic fields. There is also a limit to how much electromagnetic power one can use; since beyond that limit the samples and their contents can be damaged. The inventors have discovered that, surprisingly, adding smaller particles, polymer(s), and combinations thereof to a colloidal suspension of larger particles provides a means to reduce the repulsive interaction between any particles of interest. Surprisingly, when a second particle concentrating mechanism is applied to the sample (e.g. electromagnetic force such as a laser, electrical field, or magnetic field, or combination thereof) that addition of polymer and/or particles reduces the needed applied level of electromagnetic force to accomplish desired concentration and/or isolation of particles of interest.

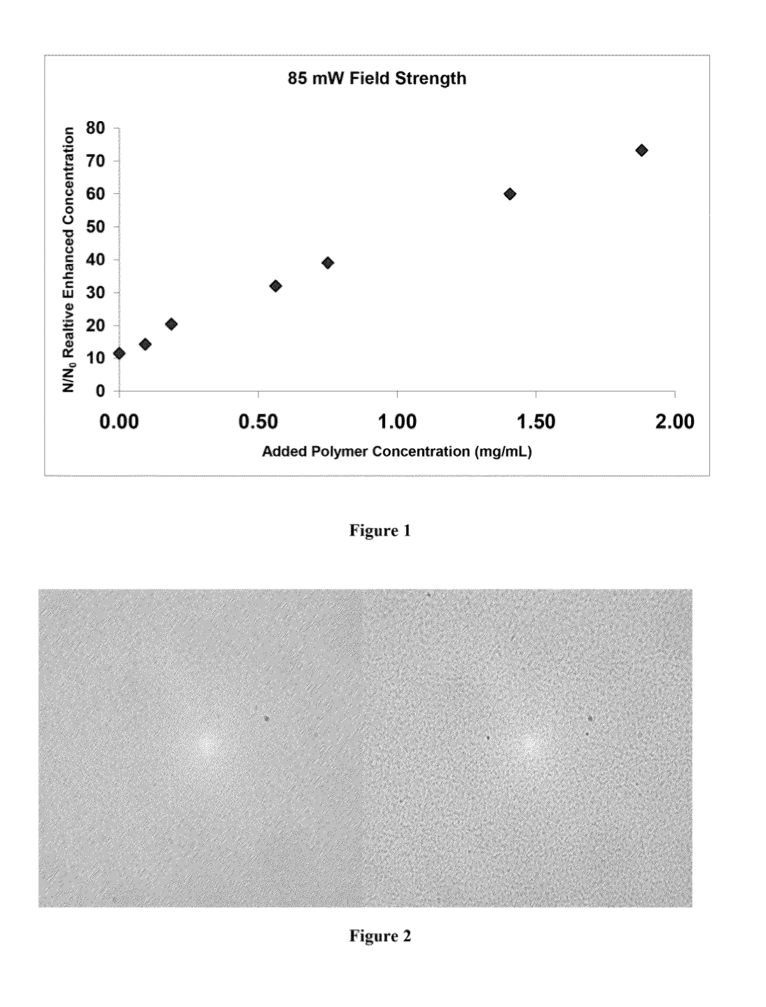

Thus, by fixing the field strength and varying the concentration of added smaller particles, we have observed increases in the large particle concentration enhancement. By way of example, the graph of FIG. 1 shows the relative increase in large particle concentration (N/N0) as a function of added small particle at a fixed laser field of about 85 mW field strength.

The advantages to using such a method are that the particle concentration enhancement can be highly localized to a predetermined area. Moreover, the concentration can be accurately controlled, and the entire phenomenon can be designed for complete reversibility.

Modifications to the controllable variables of the methods include production and variation of the field mechanism, including localized electric, magnetic, and optical fields.

Although the concept of field confinement and small particle-induced concentration enhancement have been explored separately, it is noted that our novel combination of the two provides a surprisingly greater concentration effect of the smaller particles, as well as the control of the spatial location at which the concentrating effect takes place. This novel combination yields a localized, fine-tuned mechanism for particle concentration.

Other advantages of the methods described herein include enhanced field-based concentration without damage to the test sample, as well as reversibility of the smaller particle concentration effect. Anticipated uses for the methods include the separation and diagnostics of small particles in the biotechnology industry and separation of particulate materials from suspensions in chemical industry. Exemplary particles include live bioparticles such as cells and pathogens in blood in biotechnology applications, particulate product of a chemical process, or particulate waste in water treatment plants.

Limitations of the methods relate to the particle size, since particles may exist that are too small to be affected by the at least two applied particle concentrating mechanisms would not become as highly concentrated.

The methods provided herein are suitable for concentrating particles, such as nanoparticles, microparticles, and combinations thereof in a reversible and spatially controllable manner. In a typical application, particles with two different sizes are present in the suspension; the larger particles are concentrated by the more numerous smaller ones. The concentration enhancement is primarily governed by the size difference and the relative concentrations of the two species. In one embodiment, the method uses a combination of two independent particle-concentrating mechanisms. One mechanism involves electromagnetic forces (e.g., lasers, such as a trapping laser), and the other mechanism involves polymer-induced depletion forces. Using a focused laser beam, we have demonstrated that particles can be confined and concentrated in a highly localized region. Inter-particle repulsion limits the degree to which particles can be concentrated by electromagnetic field. However, a small amount of inter-particle repulsion is needed to render colloidal stability. There is also a limit to how much electromagnetic power one can use; beyond this limit the samples may become undesirably damaged. The methods utilize the discovery that adding smaller particles or polymers to a colloidal suspension of larger particles provides a means to reduce the repulsive interaction between the particles of interest, thus alleviating the need for very high electromagnetic power (or “field strength”). Importantly, the discovery includes the polymers as a medium for suspending particles of interest as an advantage over other destabilizing agents (e.g., salts) because the colloidal stability is reversible. Without using excessive field strength that could cause irreversible damage to the sample and/or the waste of energy, the combination of these two independent particle-concentrating mechanisms provides an unprecedented control, confinement, and concentration enhancement ability. The description and figures hereof provide additional detail and disclosure concerning the novel methods and associated apparatuses.

In a first exemplary embodiment, a colloidal suspension of 190 nm diameter polystyrene particles and either 100 kg/mole molecular weight (poly)ethyleneoxide (PEO) polymers or 23 nm polystyrene particle mixtures were prepared. The suspension medium was deionized water. A vortex mixer was used to homogenize the mixture. After mixing, the suspensions were optically translucent, similar to samples without added smaller particles. The initial concentration of the 190 nm particles was 0.10% or 0.25%, or (2.8×10¹¹ or 7.1×10¹¹ particles per mL, respectively). The PEO polymer was added up until a maximum amount of 1.88 mg/mL or 1.2×10¹⁶ polymers per mL. Sample mixtures were introduced into the chamber which was a thick glass microcapillary of about 2 mm by 50 mm by 00 microns in dimension. The trapping laser (at 1064 nm wavelength) was coupled into an oil-immersion objective lens (100× magnification, 1.3 numerical aperture) forming a focal spot ˜20 microns from the microcapillary surface, where interactions arising from charges on the surfaces of the sample chamber were negligible. A second laser for fluorescence excitation (532 nm) was focused by the same objective lens and aligned parfocally with the trapping laser. Fluorescent signals emanating from the focal region were band-passed through a pinhole at location conjugated to the common focal region to ensure confocal detection of the ambient and optically-enhanced large (190 nm) particle number densities.

The system was calibrated first by measuring the fluorescence output from prepared large particle samples of known number densities in the absence of optical trapping. Once the trapping laser was turned on, the electromagnetic force produced at the focal point attracted the larger particles, thus locally increasing their concentration. This optically-induced concentration increase produced a detectable change in fluorescence output. Using the calibration, we converted the increased fluorescence output into an increase in particle concentration. In the same manner, when the smaller particles or polymers were added to the suspension, we observed an increase in fluorescence and hence concentration, above and beyond the case for only large particles in the optical field.

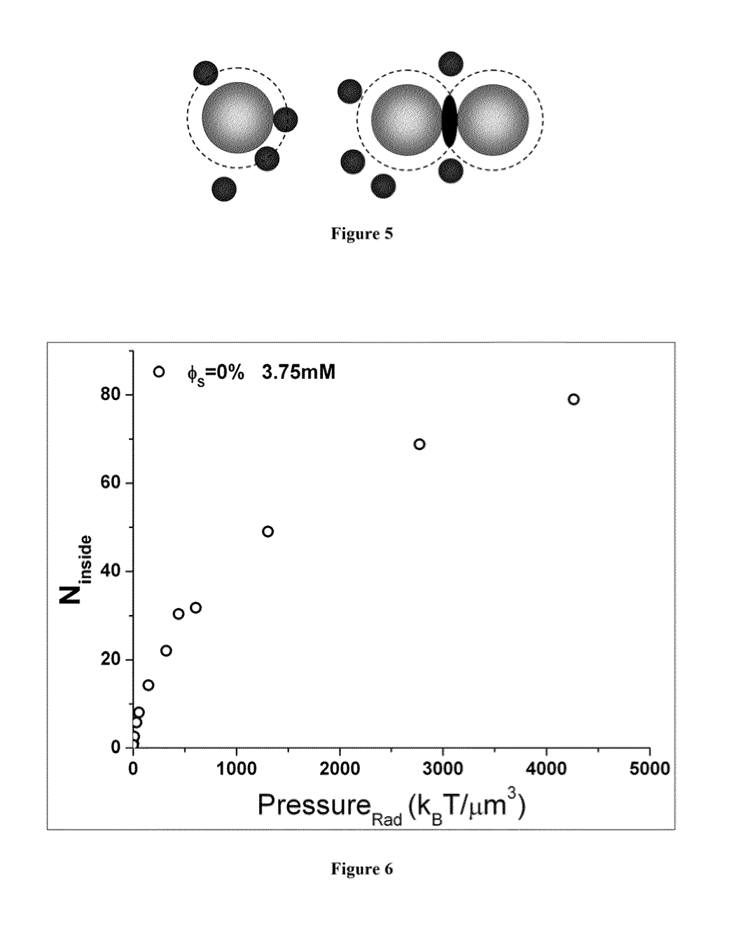

Depletion in a Mixture of Hard Spheres. As shown in FIG. 5, depletion attraction is illustrated with a hard sphere model to introduce and describe the concept of depletion. Consider a mixture of large and small hard spheres where there are no long range interactions between the large spheres and interpenetration of the particle surfaces is forbidden. For comparable volume fractions of large and small particles, there will be considerably more of the smaller spheres, than the large. As shown in FIG. 5, around each large particle there is a spherical shell, thick as the small particle radius into which the center of the smaller particles cannot penetrate. This shell reduces the total volume accessible to the small particles. In order to maximize their entropy, the smaller spheres exert an unbalanced osmotic pressure on the larger spheres, pushing them together. This forces the excluded volumes to overlap, increasing accessible volume for the smaller spheres. The volume overlap, shown in black in FIG. 5 induces attraction between the larger spheres to explain phase separation in colloid-polymer mixtures.

Optical Bottle. We first consider the laser radiation pressure exerted on the particles in the focal volume. We neglect the radiation scattering force, typically small compared to the gradient force for particles much smaller than the laser wavelength. We also assume the radiation pressure due to the gradient force to be isotropic to simplify the argument. Envisioning that the focused laser forms a trapping potential energy well of depth U₀ per particle, we equate the average radiation pressure and the average trapping energy density as: P_{rad}≈½NU₀ where N is the number density of particles in the trapping volume and the factor ½ approximately accounts for the Gaussian distribution of the laser intensity in the focal region. We use an experimentally determined factor β to relate the laser intensity and the trapping potential as U₀=βI_{Laser}. The value of β is insensitive to both the interactions between the colloidal particles as well as the presence of the added polymer, since it represents only the electromagnetic coupling of the colloidal particles to the laser. To establish the mechanical balance of the nanoparticles in the trap we consider the isothermal osmotic compressibility of the ensemble. We assert that radiation pressure causes the local particle concentration to increase, exactly balanced by the resulting excess in osmotic pressure:

$${κ}_{T} ≡ \frac{1}{N} \frac{\partial N}{\partial P} \approx \frac{Δ N}{N} \frac{1}{\overline{{P}_{rad}}} \qquad ( 1 )$$

Using the expression for the radiation pressure, we express relative enhancement of the particle number density ΔN/N in the trap as a function of the laser intensity.

$$\frac{Δ N}{N} = \frac{1}{2} {κ}_{T} {NU}_{o} = \frac{1}{2} {κ}_{T} N β {I}_{o} \qquad ( 2 )$$

The trapping potential can be experimentally extracted, and for these 190 nm polystyrene particles in water we determined β=14×10⁻¹⁰ k_{B}T (m²/W) at 8 mW of laser, or U₀=14 k_{B}T per particle. Experimentally, we measure ΔN/N as a function of laser intensity I₀ for different ambient particle concentrations.

As previously described, materials used during experimentation were 190 nm diameter polystyrene spheres (Bangs Labs) in an aqueous suspension at 1% solids (w/w). These particles were fluorescently labeled with a dye (Suncoast Yellow) having excitation and emission maxima at 540 nm and 600 nm, respectively. The polymer used was polyethylene oxide (PEO) (Sigma), having a molecular weight of 100 kg/mol and a hydrodynamic radius of 13 nm, determined by dynamic light scattering (Brookhaven Instruments). This radius was used to calculate the effective volume fraction of the PEO solutions based on the mass concentration of PEO in each sample. The polymer samples were prepared by adding pre-weighed dry powder to deionized water, and they were mixed overnight on a stirrer plate. Before being mixed with the colloid suspension, the polymer solution was vigorously sonicated to break up any aggregates before being passed through a 0.2 μm syringe filter (Millipore). The polystyrene samples were dialyzed in regenerated cellulose membrane tubes (Spectra/Por) against deionized water in the presence of a mixed-bed ion exchange resin (Dowex Marathon MR-3) for several days. The deionized 0.25% polystyrene samples were made by adding de-ionized water (Barnsted, Easy Pure RF, conductivity 0.09 μS/cm) into the solutions. For the solutions at varying ionic strength, the 190 nm solutions were dialyzed against a salt solution (NaCl, Fisher, Lot 941852) of the desired ionic strength. Polymer-colloid mixtures were made using the dialyzed aqueous 190 nm particle suspensions, the volume fraction of the colloids and the mass fraction of the polymers in each solution represent the values post-mixing. The final concentrations of PEO used in the experiment were [0.00, 0.19, 0.75, 1.88 mg/mL], corresponding to calculated volume fractions of φ_{S}=[0, 1.13, 4.65, 11.25%]. These were below the overlap concentration for polymer entanglement.

The sample chamber for the optical study was a rectangular micro-capillary (Vitrocom) attached to a microscope glass slide. Once loaded with the sample, the capillary ends were sealed with high-vacuum grease (Dow Corning). Optical trapping was achieved by a variable power IR (1064 nm) laser coupled into a high numerical aperture (NA=1.3) oil-immersion objective lens (100×, PlanFluor, Olympus). A second laser (532 nm), aligned and focused by the same objective lens to be parfocal with the trapping laser focus was used for fluorescence excitation. Fluorescent signals emanating from the focal region common to both beams were band-passed to a pinhole at a location set conjugate to the common focal region to ensure confocal detection and an optical chopper with a lock-in amplifier enhanced the signal-to-noise ratio.

Methods. Prior to using the trapping laser, the system was calibrated using the emitted fluorescence from samples containing known number densities. When the trapping laser was turned on, radiation pressure pushed particles towards the focal region, increasing the number density. This optically induced increase in number density produced a detectable increase in emitted fluorescence. Since only the large particles are fluorescent and acted on by the trap, the ability to quantify their laser-induced density changes. The calibrated system was then used to quantify the relative enhancement in the number of particles as a function of trapping intensity, (ΔN/N). Using the value for β and the data of ΔN/N as a function of I₀, the trapping intensity was converted into the optical gradient force-generated pressure from the known mean number density (inside the optical bottle) at each laser intensity as N_{inside}=N₀(1+ΔN/N). N₀=0.7 particles/μm³ were used as the ambient number density (N₀) for 190 nm particles at φ=0.25% volume fraction. In FIG. 5, the plot N_{inside} vs. ½ N_{inside}U₀ (radiation pressure converted from trapping intensity P_{rad}≈½NU₀) for 190 nm polystyrene at 0.25% with no added polymer. The ionic strength was first set to 3.75 mm to screen out repulsive electrostatic interactions for a range much less than the particle size, (κ⁻¹˜7 nm).

Results of Osmotic Compressibility of Colloid Mixtures. The radiation and osmotic pressures are proportional to the trapping intensity; therefore, the differential slope of the number density inside the optical bottle as a function of the gradient force is proportional to the isothermal compressibility, see Eq. (2), above.

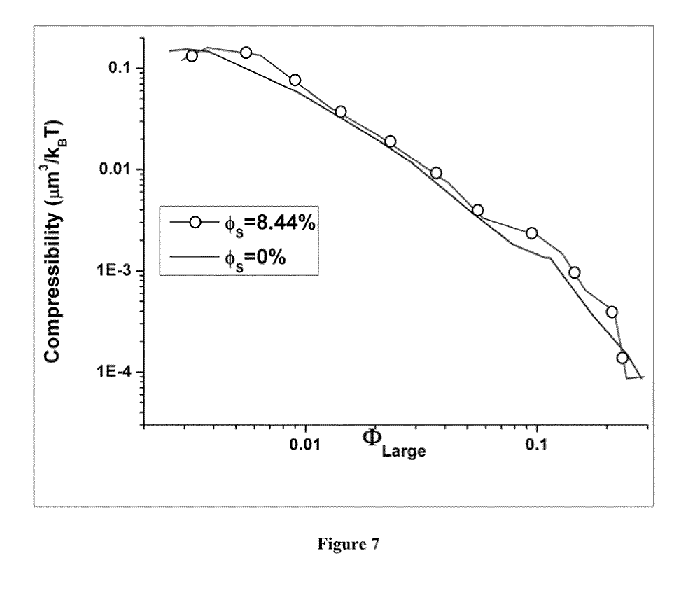

Therefore, to calculate the compressibility, the differential slope at each data point in FIG. 6 was taken and normalized the slope by the mean number density at that point. Due to the high coupling of the laser to the particles, (U₀=14 k_{B}T per 8 mW) and because the optical system was capable of trapping powers up to 80 mW, use of the optical bottle to locally enhance the number density of the colloids such that a broad concentration range could be investigated, is shown as φ_{Large}. FIG. 7 shows the osmotic compressibility as a function of volume fraction for 190 nm particles at 3.75 mM without any added polymer. Similarly, we measured the compressibility for a sample of 190 nm colloids 0.25% mixed with 8.44% polymer which is also shown in FIG. 7.

Osmotic compressibility of colloid mixtures as a function of ionic strength. As previously stated, the compressibility of colloid-polymer mixtures with the optical bottle were investigated. Regarding the compressibility of these mixtures as a function of ionic strength, pairs of samples were prepared 190 nm 0.25% colloids at different ionic strengths, both with and without 8.44% polymer.

Since the sample pairs created multiple data sets, the results in terms of the second virial coefficient are described herein. The isothermal osmotic compressibility can be expressed in terms of the virial expansion as κ⁻¹=Nk_{B}T(1+2B₂N+3B₃N²+ . . . ) where B₂ and B₃ are the second and third virial coefficients and represent the contributions from two and three-body interactions, respectively. The second virial coefficient can be calculated using the pair interaction potential U(r):

$${B}_{2} ( T ) = - 2 π {\int }_{0}^{∞} ( {e}^{- U ( r ) / {k}_{B} T} - 1 ) d {r}^{->} \qquad ( 3 )$$

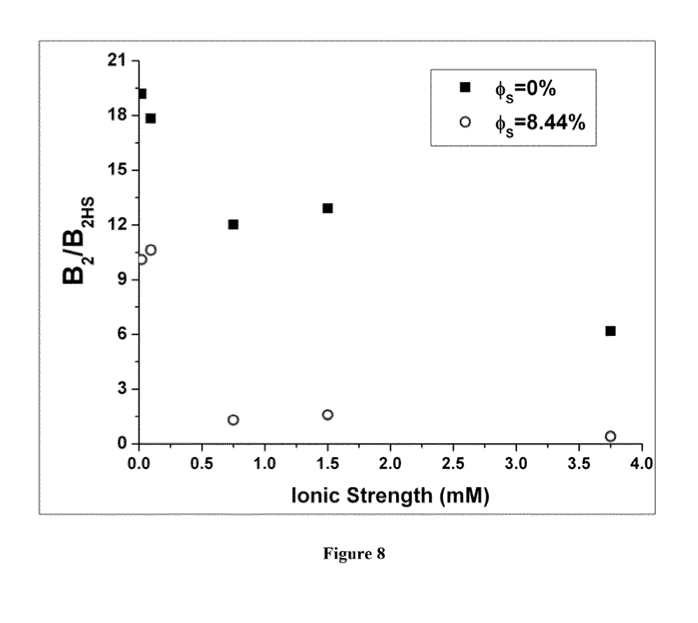

Using the virial expansion, the second virial coefficient can be extracted from the compressibility by plotting κ⁻¹ as a function of number density. Table 1 shows the extracted second virial coefficient, normalized by the hard sphere virial coefficient B_{2HS} for each of the samples as a function of ionic strength for colloid and colloid-polymer mixtures. These values are plotted in FIG. 8.

TABLE 1
The second virial coefficients, extracted from the compressibility
as a function of ionic strength for 190 nm colloids and
colloid polymer mixtures containing 8.44% PEO.

| | □_{S} = 0% | □_{S} = 8.44% |
|---|---|---|
| Salt (mM) | B₂/B_{2HS} | B₂/B_{2HS} |
| 3.75 | 6.17 | 0.4035 |
| 1.5 | 12.90 | 1.58 |
| 0.75 | 12.03 | 1.30 |
| 0.094 | 17.83 | 10.62 |
| 0.02 | 19.18 | 10.10 |

To estimate the polymer effect, the reduction in the second virial coefficient is examined by comparing the experimental values to calculated values using a simple model for the interactions in the system. The 190 nm particles are charged and interact through the screened Coulomb interaction U_{Coulomb}. When polymer is added, the particles now interact with a combined potential of U_{Total}=U_{Coulomb+}U_{Dep}. The Asakura-Oosawa (AO) model for the depletion potential is first assumed. The depletion attraction is proportional to the product of the osmotic pressure from the small particles of radius R_{g} and the excluded volume overlap of the approaching large particles of radius R. The colloid-colloid and colloid-polymer pairs cannot interpenetrate, but the polymer is free to overlap. The effective pair potential due to depletion is shown as Eq. (4).

$${U}_{Dep} ( r ) = \frac{- 4 π}{3} {d}^{2} {n}_{p} {k}_{B} T [ 1 - \frac{3 r}{4 d} + \frac{{r}^{3}}{16 {d}^{3}} ] 2 R < r < 2 d \qquad ( 4 )$$

With nₚ as the polymer number density, and d=R+R_{g} the distance of closest approach between the colloid and the polymer. For PEO with Rg˜13 nm and φ_{S}=8.44%, we deduce nₚ˜2.2×10²² m⁻³.

Using the combined potential U_{Total} and Eq. (3), we calculated the second virial coefficient for the colloid-polymer mixtures at two different ionic strengths, shown as Table 2.

TABLE 2
The second virial coefficients normalized by the hard sphere
values, as a function of ionic strength of 190 nm colloids,
with and without added 8.44% PEO. Experimental values shown
as (Exp), calculated values shown as (Calc).

| | PEO | | | |
|---|---|---|---|---|
| | □_{S} = 0% | □_{S} = 0% | □_{S} = 8.44% | □_{S} = 8.44% |
| Salt | B₂/B_{2HS} Exp | B₂/B_{2HS} Calc | B₂/B_{2HS} Exp | B₂/B_{2HS} Calc |
| 3.75 mM | 6.17 | 1.637 | 0.4035 | 1.635 |
| 1.5 mM | 12.9 | 2.323 | 1.58 | 2.323 |

To see if the estimate could be improved, the calculation was repeated with an order of magnitude increase in polymer concentration and found at 3.75 mM with nₚ˜2.2×10²³ m³ that B₂/B_{2HS}=1.51. From the results in Table 2, it is clear that the calculations underestimate the effect of the depletion when compared to the experiment. This discrepancy is more pronounced for lower ionic strength, as shown for 1.5 mM, where the presence of the polymer does not reduce the value within three decimal places. It is believed that the AO model for depletion cannot be used to calculate a qualitative estimate for the second virial coefficient using the experimental parameters described. Intrigued by this discrepancy, the depletion experiment was continued with fully deionized mixtures to see how much the polymer would change the compressibility of the colloids, if at all.

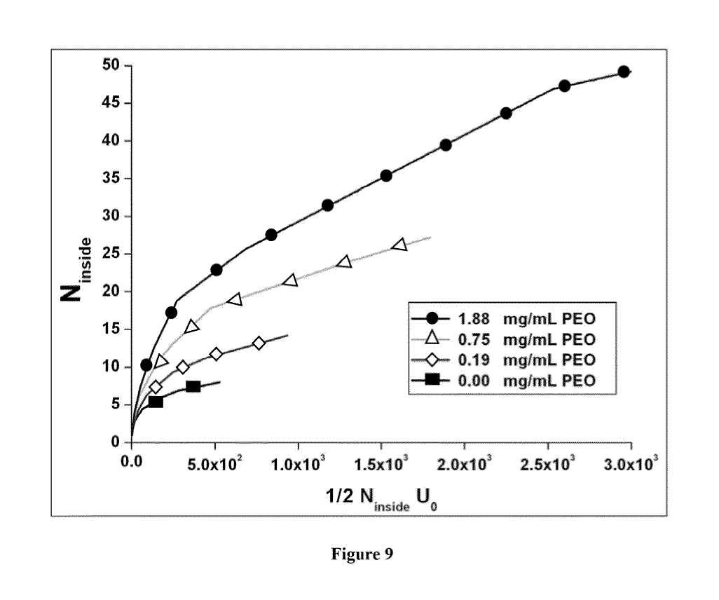

Osmotic compressibility of deionized colloid mixtures. Recently there has been growing interest in the understanding of depletion in electrostatically stabilized colloidal mixtures. Surprisingly, it has been found that long-ranged interactions between both the large-large and small-large pairs actually enhances both the range and strength of the depletion effect when compared to hard sphere interactions only. It has been suggested that the depletion potential for charged colloids becomes more complex, leading to rich phase behavior, depending on the charges on the particles and the screening length. Inspired by this effect, a simpler case was started. During experimentation, neutral polymers were mixed into a deionized aqueous suspension of 190 nm colloids. The initial assumptions were that only the large particles electrostatically repel each other, while the polymer was allowed to pass freely through the electric double layer surrounding the large colloids. Since it is assumed that the neutral polymer was unaffected by the direct colloid-colloid interactions, the expected result was that the added polymer would have little or no effect on the compressibility of the larger colloids. It was found that even for these deionized samples, the added polymer caused a marked increase in the concentrating ability of the optical bottle as shown in FIG. 9 for the same laser intensities. The x-axis is scaled by the mean radiation pressure P_{rad}˜½NβI₀ and the y-axis signifies the mean 190 nm particle number density in units of colloids/μm³.

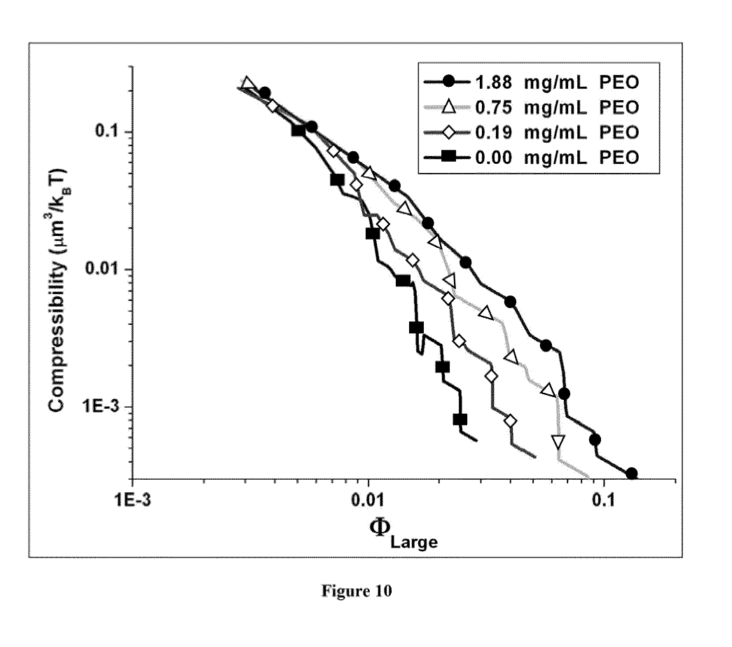

As with the high ionic strength samples, this increased number density due to added polymer indicated an enhanced large particle compressibility due to depletion effects. Using the differential slopes of FIG. 9, the osmotic compressibility was computed for the deionized samples as a function of added polymer, shown in FIG. 10.

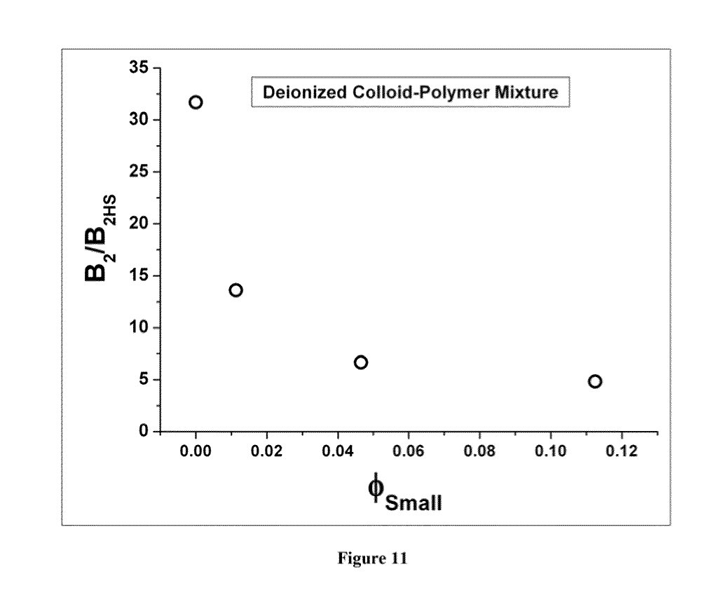

As observed in the samples with added electrolyte, adding polymer to a colloidal suspension results in an enhanced compressibility. Using the virial expansion, it is determined herein that the second virial coefficient from the deionized samples, plotted as FIG. 11.

This decrease in the second virial coefficient is presumably due to the induced depletion attraction by the polymers, but it is intriguing considering the range of the interactions in the system. The attractive depletion attraction is considered to have a range dictated by the sizes of the particles, in this case 2d=2R+2R_{g}˜218 nm. In a deionized suspension without any added electrolyte, the screening length is considerably long ˜340 nm for 1.6 μM. Thus, the thickness surrounding electric double layer around the large particles leads to fewer occurrences where the smaller particles are rejected by approaching larger particles. It seems that the repulsion should therefore prevent the depletion effect from occurring. However, as shown by FIG. 11, the second virial coefficient of this deionized mixture was continually reduced for increased polymer concentration, indicating the possibility of turning negative. For a negative second virial coefficient, the suspension starts to become unstable, but applied radiation pressure from the optical bottle could accelerate this process and promote cluster formation. Considering only the AO model, the polymer concentrations used in the experiment lower the second virial coefficient only slightly, not enough to account for the observed significant increases in compressibility. In some experiments, it was not possible to use the AO model to fully interpret the results from the deionized suspensions. Nonetheless, we believe that the combination of depletion and long range electrostatic interactions in this experiment indicate the presence of a delicate interplay between the two effects.

The effects of polymer added to a high ionic strength colloidal suspension were interpreted using the AO model. The results listed herein indicate that the AO model could not be used to interpret our data using the experimental parameters. Moreover, for decreasing ionic strength, it is found that the AO model further underestimates the depletion interaction when compared to experimental results (Table 2). The discrepancy between theory and experiment becomes larger for lower ionic strengths, as the theory predicts very little if any reduction in the second virial coefficient. Intrigued by the discrepancies, a colloid-polymer mixture is prepared at very low ionic strength. Neutral polymer added to an electrostatically stabilized dispersion would not enhance the compressibility. However, it is observed that a marked increase in the local colloid density at each addition of polymer, indicating that the compressibility had indeed increased.

The addition of polymers to a colloidal suspension changes the thermodynamic properties and structure of the suspension, through the depletion interaction. This interaction can be tuned with different sized polymers, providing control of colloidal suspensions for both industrial and scientific interests. In order to investigate the effects of depletion, an experiment was conducted to directly measure the osmotic compressibility of a colloid-polymer mixture. During the experiment multiple particles were trapped with an optical bottle and quantified the number of particles trapped as a function of laser intensity. Modeling the concentration of colloids in the optical bottle as a result of competing osmotic and radiation pressures, the experiment was able to determine the osmotic compressibility as a function of polymer concentration and solution ionic strength. This study differed from scattering or turbidity measurements of the osmotic compressibility for colloid-polymer mixtures due to the particle concentrating ability of the optical bottle. The high coupling of the large particles to the laser field permitted an investigation of the mixture across a wide range [φ_{Large}=0.25->10%] of large particle concentrations in the presence of the small polymers, eliminating some of the samples required for a large range of particle mixtures. The inventors expected that the electrostatic repulsion between the colloids would be the dominating interaction, and the depletion effect would be significantly diminished, especially in the deionized samples. However, the inventors were surprised to find that the depletion effect was still present, and resulted in an increase in the osmotic compressibility of the colloids. The attempts to compare the experimental results to a simple theory by superposition of the Coulomb and depletion potentials suggested that there is interplay between them is more complex. This showed that the optical bottle could be used to not only measure the osmotic compressibility of colloid-polymer mixtures, but function as a particle concentrator to drive a system to a desired volume fraction of the larger species. Through judicious choice of particle sizes, it should be possible to use the optical bottle to induce a phase transition through the combined effects of depletion and compressive radiation pressure.

The below additional examples are experiments demonstrating the capabilities of the polymer-mediated electromagnetic field-based particle concentrator using different combinations of large and small particle sizes. In all cases, the increased addition of smaller species acted to enhance the concentration ability of the electromagnetic field.

### Example 1

Light-induced polymer-enhanced concentration of 190 nm polystyrene particles (large species) using 100 kg/mol molecular weight (poly)-ethylene oxide (PEO) polymers as the small species. Provided was a cleaned and dialyzed suspension of 190 nm polystyrene particles in water at 0.25% volume fraction as the test species. To enhance the concentration of the test particles, we added varied concentrations of PEO [0.00-1.88 mg/mL]. As the concentration of the small particles was increased, we observed a marked increase in the concentration-enhancement of the larger particles at a constant optical field strength. In this example, the PEO polymers and polystyrene particles had little or no mutual electrostatic repulsion.

### Example 2

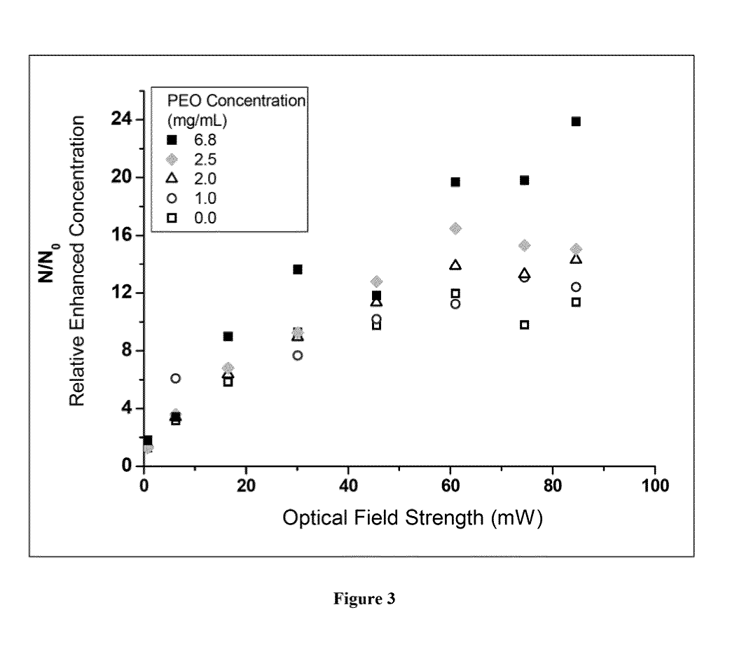

Light-induced polymer-enhanced concentration of 190 nm polystyrene particles (large species) by the use of 23 nm polystyrene particles as the small species. We used a cleaned and dialyzed suspension of 190 nm polystyrene particles in water at 0.10% volume fraction as the test species. To enhance the concentration of the test particles, we added varied concentrations of 23 nm polystyrene particles (1% to 9% volume fraction) to the 190 nm particle sample, shown in FIG. 2. In the right of FIG. 2, it is noticeable that phase separation is about to take place, even without the optical field. Thus, the optical field can accelerate or even magnify the phase separation phenomenon as shown in FIGS. 1 and 3. In this example, there is a significant mutual electrostatic repulsion as both the large and small particles were composed of polystyrene, which are known to develop a negative surface charge when dispersed in water.

### Example 3

Light-induced polymer enhanced concentration of 210 nm polystyrene particles (large species) using 100 kg/mol molecular weight (poly)-ethylene oxide (PEO) as the small species.

### Example 4

Light-induced polymer enhanced concentration of 100 nm polystyrene particles (large species) using 20 kg/mol molecular weight (poly)-ethylene glycol (PEG) as the small species as a function of laser intensity, shown in FIG. 3.

### Experimental Details

The experiments were performed with different sizes of large and small particles and/or at different concentrations of added polymers. Details for a typical experiment (190 nm polystyrene particles mixed with PEO polymer) are shown below. However, the methods are applicable to any mixture of particles wherein the ratio of particle size of the large particles to the small particles is greater than about 3:1, and more preferably greater than about 5:1, and most preferably greater than about 8:1. Further, the methods have been shown to work for mixtures where the large particles and small particles are nanosized, and in other examples where the small particles are less than 50 nm and the large particles are particles greater than about 190 nm. A number of bioparticles fall within those size ranges, and particularly in the larger particle size range stated herein. Bioparticles are subject to the same repulsion and other forces described herein, and therefore being controllable and concentratable using the methods herein when a biocompatible polymer is used as the suspension medium.

A colloidal suspension of fluorescently labeled 190 nm diameter polystyrene particles and either (poly) ethylene oxide polymers or the smaller (23 nm diameter) polystyrene particle mixtures were prepared. The suspension medium was deionized water. A vortex mixer was used to homogenize the mixture. After mixing, the suspensions were translucent, similar to samples without added smaller particles. The initial concentrations of the 190 nm particles were 0.10% or 0.25%, or (2.8×10¹¹ or 7.1×10¹¹ particles per mL, respectively). The PEO polymer was added up until a maximum amount of 1.88 mg/mL or 1.2×10¹⁶ polymers per mL. Sample mixtures were introduced into the chamber previously described herein. The trapping laser (1064 nm in wavelength) was coupled into an oil-immersion objective lens (100× magnification, 1.3 numerical aperture) forming a focal spot ˜20 microns from the microcapillary surface, where interactions arising from charges on the inner surfaces of the sample chamber were negligible. A second laser (532 nm in wavelength) for excitation of the fluorescent labels within the 190 nm particles was focused by the same objective lens and aligned parfocally with the trapping laser. Fluorescent signals emanating from the focal region were band-passed through a pinhole at a location conjugated to the common focal region to ensure confocal detection of the ambient and optically-enhanced large (190 nm) particle number densities.

The system was calibrated first by measuring the fluorescence output from prepared large particle samples of known concentrations in the absence of a trapping laser. Once the trapping laser was turned on, the electromagnetic force produced in the focal point attracted the larger particles, thus locally increasing their concentration. This optically induced concentration increase produced a detectable increase in fluorescence output. Using the calibration, we converted the increase in fluorescence output into increase in particle concentration. In the same manner, when the smaller particles or polymers were added to the suspension, we observed an increase in fluorescence and hence concentration, above and beyond the case for only optical field-induced concentration of the large particles.

### Example 5

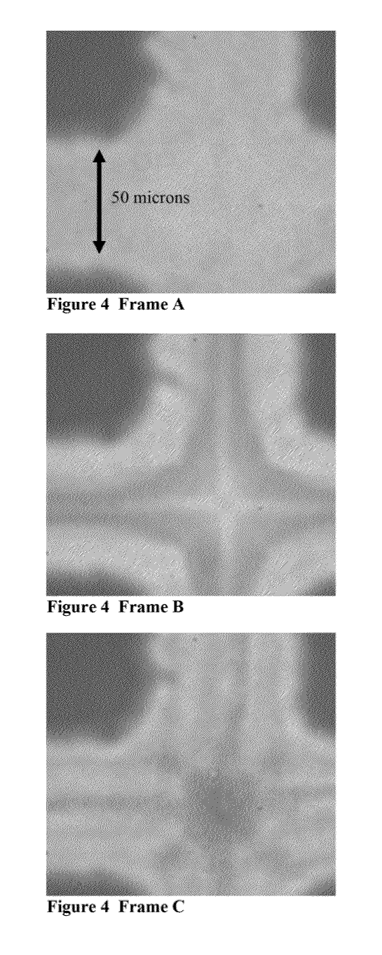

In addition to localized optical fields, we have also demonstrated that enhancing the particle concentration can be accomplished with localized electric fields. For colloidal particles in suspension in a radial-frequency AC electric field, the relaxation of the ionic species in the shear layer gives rise to a frequency-dependent dielectrophoretic (DEP) force that drives the particles along the direction of field intensity gradient. This DEP force, analogous to the optical trapping force but operates at different frequencies, may be used to concentrate particles. Thus, the polymer-mediated reduction in repulsion between the particles still enhances the ability of the electric field to locally concentrate particles. In FIG. 4, illustrated is data from an experiment involving 190 nm polystyrene particles mixed with PEO polymers, but acted on by a focused electric field created by gold-film electrodes (shown as dark regions on the corners of the region shown in FIG. 4) coated on a glass substrate. The sample consisted of 190 nm polystyrene particles at 0.5% volume fraction mixed with 1.88 mg/mL PEO. Fluorescence detection was also used to monitor the concentration of large particles (190 nm) as a function of electric field strength and added polymer.

By way of further example, the Figures herein provide supporting and additional disclosure. FIG. 1 is a graph representing data showing the relative concentration enhancement as a function of added polymer, at a fixed electromagnetic field strength.

FIG. 2 illustrates 190 nm polystyrene particles mixed with 23 nm polystyrene particles before an optical confinement field is administered.

FIG. 3 is a graph depicting the concentration enhancement of 210 nm polystyrene particles mixed with different PEO (polyethylene-oxide) polymer concentrations, as a function of optical field strength.

FIG. 4 illustrates an enhanced concentration of 190 nm polystyrene latex particles mixed with PEO polymers in a localized electric field. Sequential frames depict the localized concentration as a function of time once a non-uniform electric field is turned on.

FIG. 5 is an illustration of depletion attraction of a hard sphere model. Specifically, on the left, the center of each small particle is excluded from a layer of radius Rg around each large particle. On the right, illustration of the layers overlapping, which creates more volume accessible to the smaller spheres.

FIG. 6 is a graph illustrating the mean 190 nm particle number density inside the optical bottle as a function of optical gradient-generated pressure.

FIG. 7 is a graph illustrating the osmotic compressibility for 190 nm colloids at 3.75 mm, shown for 0% and 8.44% added polymer concentrations, calculated from the data in FIG. 6.

FIG. 8 is a graph illustrating the second virial coefficients shown in Table 1, plotted as a function of ionic strength for 190 nm colloids, with and without added 8.44% PEO.

FIG. 9 is a graph illustrating the mean 190 nm particle number density inside the optical bottle as a function of optical gradient-generated pressure for samples containing different concentrations of polymer, data taken using identical laser intensities.

FIG. 10 is a graph illustrating the osmotic compressibility for deionized 190 nm colloids as a function of volume fraction shown at different added polymer concentrations.

FIG. 11 is a graph illustrating the second virial coefficient determined from the compressibility plots in FIG. 10. Data is shown for the deionized samples of 190 nm colloids as a function of added polymer.

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, in the description there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

1. A method for concentrating a sample, the method comprising the steps of:
providing a sample comprising particles, the particles being provided in at least two different sizes, the sample further comprising a polymer suitable for suspension of the particles;
applying a first particle concentrating mechanism; and
applying a second particle concentrating mechanism,
wherein at least one of the particle concentrating mechanisms involves electromagnetic force, and wherein at least one of the particle concentrating mechanisms involves polymer-induced depletion force.

2. The method of claim 1, wherein the particles comprise at least some nanoparticles, and further comprise at least some microparticles.

3. The method of claim 2, wherein the electromagnetic forces are applied by at least one of electric, magnetic, electromagnetic field, and combinations thereof.

4. The method of claim 2, wherein the electromagnetic forces are applied in a manner to avoid damage to the sample.

5. The method of claim 1, wherein the combination of the first particle concentrating mechanism and the second particle concentrating mechanism overcomes the inter-particle repulsion forces that would otherwise limit the degree to which the particles can be concentrated by electromagnetic force alone.

6. The method of claim 5, wherein the step of applying a first particle concentrating mechanism and the step of applying a second particle concentrating mechanism overlap in time.

7. The method of claim 6, wherein the first particle concentrating mechanism comprises electromagnetic force, and wherein the second particle concentrating mechanism comprises polymer induced mediated force.

8. The method of claim 5, wherein the step of applying a first particle concentrating mechanism is commenced before the step of applying a second particle concentrating mechanism.

9. The method of claim 8, wherein the step of applying a first particle concentrating mechanism continues during the step of applying a second particle concentrating mechanism.

10. The method of claim 9, wherein the first particle concentrating mechanism comprises electromagnetic force, and wherein the second particle concentrating mechanism comprises polymer induced mediated force.

11. The method of claim 1, wherein the combination of particle concentrating mechanisms produces reversible colloidal stability.

12. The method of claim 1, further comprising the step of, after the step of applying the first particle concentrating mechanism, adding at least one of additional particles or additional polymer.

13. The method of claim 12, wherein the step of adding at least one of additional particles or additional polymer results in the use of a lower level of electromagnetic power than would otherwise be required to achieve a desired concentration of the particles.

14. The method of claim 1, wherein the method produces reversible colloidal stability in the sample.

15. The method of claim 14, further comprising the step of, after applying at least one of the first particle concentrating mechanism or second particle concentrating mechanism, turning off at least one of the mechanisms to return the sample to a lower concentration.

16. The method of claim 15, wherein the method does not degrade the quality of the sample.

17. The method of claim 1, wherein the polymer is at least one of polyethylene oxide, polyethylene glycol, a water-soluble polymer, and combinations thereof.

18. The method of claim 17, wherein the polymer is biocompatible, and wherein at least some of the particles are bioparticles.

19. The method of claim 18, wherein the bioparticles comprise at least one of viruses, bacteria, proteins, DNA, RNA, biological cells, and combinations thereof.

20. The method of claim 19, wherein the method does not damage, kill, lyse, or otherwise irreversibly alter the particles.
